(19) United States
(12) Patent Application Publication
(10) Pub. No.: US 2025/0250597 A1
(43) Pub. Date: Aug. 7, 2025

(54) EQUOL MANUFACTURING METHOD

(71) Applicant: Daicel Corporation, Osaka-shi, Osaka (JP)

(72) Inventor: Hideki MURATA, Tokyo (JP)

(73) Assignee: Daicel Corporation, Osaka-shi, Osaka (JP)

(21) Appl. No.: 18/855,898

(22) PCT Filed: Apr. 12, 2023
(86) PCT No.: PCT/JP2023/014802

§ 371 Date: Oct. 10, 2024

(30) Foreign Application Priority Data
Apr. 13, 2022 (JP) 2022-066081

(51) Int. Cl. C12P 17/06

(52) U.S. Cl. CPC C12P 17/06

(57) ABSTRACT

The present disclose provides an equol manufacturing method for producing equol including fermenting at least one type of equol raw material selected from the group consisting of daidzein glycoside, daidzein, and dihydrodaidzein.

### TECHNICAL FIELD

The present invention relates to a method for manufacturing equol by a microorganism having equol-producing capability.

### BACKGROUND ART

Isoflavones, which are abundantly contained in leguminous plants such as soybeans and kudzu, are flavonoids that are a class of polyphenols and have an isoflavone backbone. Recent studies have revealed that isoflavones have a female hormonal action (estrogen) and an antioxidant action, and that the ingestion of isoflavones has a preventive effect on breast cancer, prostate cancer, osteoporosis, hypercholesterolemia, heart disease, menopausal issues, and the like.

Isoflavones are present in, for example, soybeans in the form of glycosides covalently bonded to sugars, such as daidzin, glycitin, and genistin, and are present in very small amounts in the form of aglycones. These glycosides may be further malonylated or acetylated and present in the malonylated or acetylated form. When these glycosides enter a human or animal body, the glycosides are converted to daidzein, glycitein, and genistein by the action of digestive enzymes or β-glucosidase, which is an enzyme produced by enterobacteria. Furthermore, it is known that daidzein is enzymatically converted to O-desmethylangolensin (0-DMA) or equol via dihydrodaidzein by the action of enterobacteria.

Equol is known to have the highest estrogenic activity among these metabolites. However, in humans, there are individual differences in the metabolism of isoflavones, and it is clear that few people possess enterobacteria having the ability to ferment daidzein to produce equol as described above. The prevalence rate of such enterobacteria is approximately 50% among Japanese people and approximately 30% among European and American populations. People who do not possess equol-producing bacteria cannot produce equol in their bodies even with the consumption of leguminous food such as soybeans, and therefore this inability to produce equol is an issue to be addressed.

To overcome these problems, attempts have been made to extracorporeally produce equol using anaerobic microorganisms such as lactic acid bacteria (Patent Documents 1 to 4). However, it has not been clarified what fermentation conditions and fermentation methods can manufacture equol more effectively and practically. For example, there has been an example in which four types of anaerobic microorganisms are mixed and cultured in a hydrogen gas phase to attempt to manufacture equol (Non-Patent Literature 1); however, the mixed culture is not suitable for practical production (industrialization).

Since anaerobic microorganisms are cultured, stationary culture is usually performed (Patent Document 5). On the other hand, regarding a method by which equol can be more effectively and practically manufactured by fermentation conditions and a fermentation method, there is a report that a manufacturing efficiency of equol is dramatically increased when a mass percent concentration of hydrogen gas in a gas phase in which fermentation is performed is from 40% to 100% (particularly 100%) (Patent Document 6). However, when hydrogen ignites in the coexistence of oxygen, a severe explosion occurs. In mixing with air, a hydrogen concentration range in which an explosion occurs is a lower limit of hydrogen concentration of 4.10% and an upper limit of 74.2%. That is, in the hydrogen concentration range in which the manufacturing efficiency of equol is dramatically increased, when a gas containing hydrogen leaks from a manufacturing facility, the hydrogen concentration has a possibility of causing a severe explosion; therefore, at the time of industrialization, a very expensive production facility sufficiently provided with explosion prevention measures is required.

### CITATION LIST

### Patent Document

Patent Document 1: JP 2006-204296 A
Patent Document 2: JP 2006-504409 T
Patent Document 3: JP 2008-61584 A
Patent Document 4: JP 2010-104241 A
Patent Document 5: WO 2007/066655
Patent Document 6: WO 2012/033150

### Non-Patent Literature

Non-Patent Literature 1: Decroos, K. et al., Arch Microbiol., 183, 45-55 (2005)

### SUMMARY OF INVENTION

### Technical Problem

The present invention has been made in view of such circumstances, and an object of the present invention is to provide a method capable of effectively manufacturing equol at a lower hydrogen concentration, which is safer than before, in the manufacturing of equol using microorganisms.

### Solution to Problem

The present inventor has found that, in a process of fermenting at least one type of equol raw material selected from the group consisting of a daidzein glycoside, a daidzein, and a dihydrodaidzein under a gas phase including one or more types of gases containing hydrogen by a microorganism having an ability to assimilate the equol raw material to produce equol, by using a fermenter having a culture solution in an amount of 100 L or more and, using a stirring power of a certain level or more and/or a gas supply rate condition of a certain level or more with a certain sparger, there is no limitation on the type of bacteria, and equol is effectively produced at a lower gas phase hydrogen concentration than the known one. Specifically, the following invention has been found.
<1> An equol manufacturing method for producing equol including fermenting at least one type of equol raw material selected from the group consisting of daidzein glycoside, daidzein, and dihydrodaidzein under a gas phase including one or more types of gases containing hydrogen by a microorganism having an ability to assimilate the equol raw material to produce equol, wherein in the fermentation, when a fermenter having a culture solution in an amount of 100 L or more is used, and
   i) stirring is performed at a stirring power of 0.1 kW/kL or more, preferably 0.2 kW/kL or more, more preferably 0.4 kW/kL or more in the fermenter; and/or
   ii) the one or more types of gases are introduced by using a sparger having a pore size of 2 mm or less, preferably 1 mm or less, more preferably 0.5 mm or less.
<2> In the above <1>, the one or more types of gases may contain hydrogen at a concentration of 30% or less, preferably 10% or less, and more preferably 4% or less.

### Advantageous Effects of Invention

According to the present invention, it is possible to provide a method capable of effectively manufacturing equol at a lower hydrogen concentration, which is safer than before, in the manufacturing of equol using microorganisms.

In particular, according to the present invention, it is possible to realize an efficient method for manufacturing equol using daidzein as a raw material using microorganisms, particularly anaerobic microorganism fermentation, at a low hydrogen concentration that is safe even on an industrial scale, and to provide a technique for mass manufacturing of equol.

The method of the present invention makes it possible to manufacture equol inexpensively and in large quantities, and to supply equol to more people. It is considered that equol can prevent breast cancer, prostate cancer, osteoporosis, hypercholesterolemia, heart disease, menopause disorder, and the like by directly ingesting equol as food and drink, medicine, or the like.

### DESCRIPTION OF EMBODIMENTS

The present invention provides an equol manufacturing method for producing equol including fermenting at least one type of equol raw material selected from the group consisting of daidzein glycoside, daidzein, and dihydrodaidzein, under a gas phase including one or more types of gases containing hydrogen, by a microorganism having an ability to assimilate the equol raw material to produce equol. In this method, in the fermentation, when a fermenter having a culture solution in an amount of 100 L or more is used, and
i) stirring is performed at a stirring power of 0.1 kW/kL or more, preferably 0.2 kW/kL or more, more preferably 0.4 kW/kL or more in the fermenter; and/or
ii) the gas is introduced by using a sparger having a pore size of 2 mm or less, preferably 1 mm or less, more preferably 0.5 mm or less.

Although the manufacturing of equol by metabolism of microorganisms, particularly anaerobic microorganisms, has been known for a long time from the academic viewpoint, the method of the present invention is useful because the culture conditions of anaerobic microorganisms that manufacture equol at a low hydrogen concentration that is safe even on an industrial scale have not been established.

The present invention will be described below in detail.

### Fermentation

The present invention provides a method for manufacturing equol including the fermentation.

The method according to the present invention may include a process besides the fermentation. Examples thereof include, but are not limited to, a process of preparing an equol raw material and a process of recovering the obtained equol.

Specific examples of the process other than the fermentation include, but are not limited to, a sterilization process of sterilizing microorganisms used for fermentation.

### Equol Raw Material

In the method of the present invention, particularly in the fermentation of the method of the present invention, at least one type of equol raw material selected from the group consisting of daidzein glycoside, daidzein, and dihydrodaidzein is used as a raw material.

The equol raw material may be in any form as long as the material can literally be used as a raw material for equol.

The equol raw material may be in any form as long as the equol contains at least one selected from the group consisting of daidzein glycoside, daidzein, and dihydrodaidzein. Examples of the equol raw material include daidzein glycoside itself, daidzein itself, and dihydrodaidzein itself, as well as raw materials containing these, such as soybeans, processed soybeans, soybean hypocotyls, and processed soybean hypocotyls (e.g., soybean extracts, soybean hypocotyl extracts, and purified soybean hypocotyl extracts), and specifically, commercially available isoflavones may be used.

### Microorganism

In the method of the present invention, particularly in the fermentation in the method of the present invention, a microorganism having the ability to assimilate an equol raw material to produce equol is used. The “ability to assimilate an equol raw material to produce equol” may be simply referred to herein as an “equol-producing capability”.

The microorganism that assimilate equol and used in the method of the present invention is not particularly limited as long as the microorganism is one having the ability to produce equol from the above-described equol raw material.

Examples of the microorganism include anaerobic microorganisms. The anaerobic microorganisms can produce equol at a temperature of, for example, around 37° C. (e.g., from 30 to 42° C.).

The equol-producing capability can be confirmed by quantitatively determining the daidzein, dihydrodaidzein, equol, and the like in the culture. A person skilled in the art can carry out these quantitative determinations on the basis of the descriptions of, for example, WO 2012/033150, JP 2012-135217 A, JP 2012-135218 A, and JP 2012-135219 A. An example of these quantitative determination methods is described below.

For example, ethyl acetate is added to a culture solution, the mixture is vigorously stirred and then centrifuged, and the ethyl acetate layer is extracted. The same operation can be carried out several times on the same culture solution as necessary, and the extracted ethyl acetate layers can be combined to produce a liquid extract of equol. The liquid extract is concentrated and dried under reduced pressure using an evaporator, and then dissolved in methanol. The resulting solution is filtered using a membrane such as a polytetrafluoroethylene (PTFE) membrane to remove insoluble matter, and the resulting product can be used as a sample for high performance liquid chromatography. Examples of the conditions for high performance liquid chromatography include, but are not limited to, the following.

### Conditions for High Performance Liquid Chromatography

Column: Phenomenex Luna 5uC18, 2.0 mm×150 mm (Shimadzu Glc Ltd.)
Mobile phase: water/methanol [55:45, v/v]
Flow rate: 0.2 mL/min
Column temperature: 40° C.
Detection: UV 280 nm
Retention time: 13.8 minutes for dihydrodaidzein, 19.6 minutes for daidzein, 22.5 minutes for glycitein, 25.6 minutes for equol, 35.0 minutes for genistein

Examples of microorganisms having the ability to produce equol include, but are not limited to, microorganisms classified into the following genera.
Genus Adlercreutzia
Genus Bacteroides
Genus Bifidobacterium
Genus Clostridium
Genus Eggerthella
Genus Enterococcus
Genus Enterorhabdus
Genus Eubacterium
Genus Finegoldia
Genus Lactobacillus
Genus Lactococcus
Genus Paraeggerthella
Genus Pediococcus
Genus Proteus
Genus Sharpea
Genus Slackia
Genus Streptococcus
Genus Veillonella

Specific examples of microorganisms having the ability to produce equol include, but are not limited to, the following microorganisms.
Adlercreutzia equolifaciens subsp. celatus
Adlercreutzia equolifaciens subsp. equolifaciens
Bacteroides ovatus
Bifidobacterium breve
Bifidobacterium longum
Clostridium sp.
Eggerthella sp.
Enterococcus faecalis
Enterococcus faecium
Enterorhabdus mucosicola
Eubacterium sp.
Finegoldia magna
Lactobacillus fermentum
Lactobacillus mucosae
Lactobacillus paracasei
Lactobacillus plantarum
Lactobacillus rhamnosus
Lactobacillus sp.
Lactococcus garvieae
Lactococcus sp.
Paraeggerthella sp.
Pediococcus pentosaceus
Proteus mirabilis
Sharpea azabuensis
Slackia equolifaciens
Slackia isoflavoniconvertens
Slackia sp.
Streptococcus constellatus
Streptococcus intermedius
Veillonella sp.

Examples of the above-described microorganisms include microorganisms classified into the Eggerthellaceae family, microorganisms classified into the Bifidobacteriaceae family, microorganisms classified into the Clostridiaceae family, microorganisms classified into the Coriobacteriaceae family, microorganisms classified into the Enterococcaceae family, microorganisms classified into the Eubacteriaceae family, microorganisms classified into the Morganellaceae family, microorganisms classified into the Peptoniphilaceae family, microorganisms classified into the Lactobacillaceae family, microorganisms classified into the Streptococcaceae family, microorganisms classified into the Veillonellaceae family, and related microorganisms thereof. Preferable microorganisms are those classified into the genus Adlercreutzia, the genus Bacteroides, the genus Bifidobacterium, the genus Clostridium, the genus Coriobacterium, the genus Eggerthella, the genus Enterococcus, the genus Eubacterium, the genus Finegoldia, the genus Lactobacillus, the genus Lactococcus, the genus Paraeggerthella, the genus Pediococcus, the genus Proteus, the genus Sharpea, the genus Slackia, the genus Streptococcus, and the genus Veillonella, or related microorganisms thereof. More preferable microorganisms are Adlercreutzia equolifaciens subsp. celatus, Adlercreutzia equolifaciens subsp. equolifaciens, Bacteroides ovatus, Bifidobacterium breve, Bifidobacterium longum, Clostridium sp., Eggerthella sp., Enterococcus faecalis, Enterococcus faecium, Enterorhabdus mucosicola, Eubacterium sp., Finegoldia magna, Lactobacillus fermentum, Lactobacillus intestinalis, Lactobacillus mucosae, Lactobacillus paracasei, Lactobacillus plantarum, Lactobacillus rhamnosus, Lactobacillus sp., Lactococcus garvieae, Lactococcus sp., Paraeggerthella sp., Pediococcus pentosaceus, Proteus mirabilis, Sharpea azabuensis, Slackia equolifaciens, Slackia isoflavoniconvertens, Slackia sp., Streptococcus constellatus, Streptococcus intermedius, and the Veillonella sp.

Among the above-described microorganisms, particular examples of more preferable anaerobic microorganisms include any of the microorganisms described below or related bacteria having the same species properties as these microorganisms.
Adlercreutzia equolifaciens subsp. celatus DSM 18785 strain
Adlercreutzia equolifaciens subsp. equolifaciens DSM 19450 strain
Bacteroides ovatus E-23-15 strain
Bifidobacterium breve ATCC 15700 strain
Bifidobacterium longum BB536 strain
Clostridium sp. HGH136 strain
Eggerthella sp. Julong 732 strain
Eggerthella sp. YY7918 strain
Eggerthella sp. D1 strain
Enterococcus faecalis INIA P333 strain
Enterococcus faecium EPI1 strain
Enterorhabdus mucosicola Mt1B8 strain
Eubacterium sp. D2 strain
Finegoldia magna EPI3 strain
Lactobacillus fermentum DPPMA114 strain
Lactobacillus intestinalis KTCT13676BP strain
Lactobacillus mucosae EPI2 strain
Lactobacillus paracasei JS1 strain
Lactobacillus plantarum DPPMA24W strain
Lactobacillus plantarum DPPMASL33 strain
Lactobacillus rhamnosus DPPMAAZ1 strain
Lactobacillus rhamnosus INIA P540 strain
Lactobacillus sp. Niu-016 strain
Lactococcus garvieae 20-92 strain
Paraeggerthella sp. SNR40-432 strain
Pediococcus pentosaceus CS1 strain
Proteus mirabilis LH-52 strain
Sharpea azabuensis ST18 strain
Slackia equolifaciens strain DSM 24851 strain
Slackia isoflavoniconvertens DSM 22006 strain
Slackia sp. FJK1 strain
Slackia sp. NATTS strain
Slackia sp. YIT11861 strain
Slackia sp. TM-30 strain
Streptococcus constellatus E-23-17 strain
Streptococcus intermedius A6G-225 strain
Veillonella sp. EP strain

Note that the abovementioned anaerobic microorganisms are available from the depository indicated by the deposit number. Each accession number indicates that the anaerobic microorganism is deposited in one of the following depositories.
FERM: International Patent Organism Depositary (IPOD) http://unit.aist.go.jp/pod/ci/index.html
DSM: German Collection of Microorganisms and Cell Cultures (DSMZ) http://www.dsmz.de/
KCCM: Korean Culture Center of Microorganisms

In the present invention, the anaerobic microorganism capable of producing equol is cultured under conditions suitable for the production of equol. In the present invention, the conditions suitable for the production of equol refer to conditions under which the survival and activity of the anaerobic microorganism having equol-production activity are maintained. More specifically, the conditions thereof refer to conditions under which the gas phase conditions (anaerobic conditions) in which anaerobic microorganisms can survive are maintained, and nutrients for supporting the activity and growth of the anaerobic microorganisms are provided. Various culture medium compositions suitable for the survival of the anaerobic microorganisms are known. Therefore, a person skilled in the art can select an appropriate culture medium composition for an above-described anaerobic microorganism having the ability to produce equol. For example, a BHI culture medium available from Difco Laboratories Inc. or a culture medium used in the examples can be used.

A water-soluble organic material can be added as a carbon source to the culture medium used in the present invention. Examples of the water-soluble organic material include, but are not limited to, the following compounds:
saccharides such as sorbose, fructose, and glucose; alcohols such as methanol; and organic acids such as valeric acid, butyric acid, propionic acid, acetic acid, and formic acid, or salts thereof.

The concentration of the organic material added to the culture medium as a carbon source can be adjusted, as appropriate, to efficiently grow anaerobic microorganisms in the culture medium.

A nitrogen source can be added to the culture medium. Various nitrogen compounds that can be ordinarily used in fermentation can be used as the nitrogen source in the present invention. Preferred inorganic nitrogen sources include ammonium salts and nitrates. More preferable inorganic nitrogen sources include ammonium sulfate, ammonium chloride, ammonium phosphate, ammonium hydrogen phosphate, potassium nitrate, and sodium nitrate. Meanwhile, examples of preferred organic nitrogen sources include amino acids, yeast extracts, peptones, meat extracts, liver extracts, and digested serum powder. Examples of more preferred organic nitrogen sources include arginine, cysteine, cystine, citrulline, lysine, yeast extracts, and peptones.

Furthermore, other organic materials or inorganic materials suited for the production of equol can also be added to the culture medium in addition to the carbon source and the nitrogen source. For example, in some cases, the growth and activity of anaerobic microorganisms can be enhanced by adding cofactors such as vitamins or inorganic compounds such as various salts to the culture medium. Examples of inorganic compounds, vitamins, and plant- and animal-derived cofactors for microbial growth include the following.

| Inorganic Compounds | vitamins |
|---|---|
| Potassium dihydrogen phosphate | biotin |
| Magnesium sulfate | folic acid |
| Manganese sulfate | pyridoxine |
| Sodium chloride | thiamine |
| Cobalt(II) chloride | riboflavin |
| Calcium chloride | nicotinic acid |
| Zinc sulfate | pantothenic acid |
| Copper sulfate | vitamin B12 |
| Alum | thioctic acid |
| Sodium molybdate | p-aminobenzoic acid |
| Potassium chloride | |
| Boric acid and the like | |
| Nickel(II) chloride | |
| Sodium tungstate | |
| Sodium selenate | |
| Ammonium iron(II) sulfate | |

A typically known technique can be used as the method for producing a culture solution by adding these inorganic compounds, vitamins, or growth cofactors. The culture medium can be a liquid, a semi-solid or a solid. In the present invention, the preferred form of the culture medium is a liquid culture medium.

The culture medium according to an embodiment of the present invention may contain dextrins. When an anaerobic microorganism is cultured in a culture medium containing dextrins, a liquid containing equol and dextrins can be prepared without bringing the dextrins into contact with the culture after the culturing.

The dextrins can be added to the culture medium before or during the culturing of the microorganisms.

The culture medium according to an embodiment of the present invention may contain an antifoaming agent, preferably soybean oil, and more preferably soybean oil containing vitamin E.

In the method of the present application, microorganisms, and particularly anaerobic microorganisms can be cultured according to a known method of culturing microorganisms. In industrial production, a continuous cultivation system (continuous fermentation system) that can continuously feed the culture medium and a gaseous substrate and is provided with a mechanism for recovering the culture can also be used.

When anaerobic microorganisms are used in the method according to an embodiment of the present invention, it is preferable to prevent oxygen from entering the fermenter. As the fermenter, a commonly used fermenter can be used as is. An anaerobic atmosphere can be created by replacing oxygen that mixes into the fermenter with an inert gas such as nitrogen.

Gas phase in fermentation The method of the present invention, particularly the fermentation in the method of the present invention, is carried out under a gas phase including one or more gases containing hydrogen. The gas constituting the gas phase is not particularly limited as long as the gas is composed of one or more types of gases including hydrogen, but the gas phase preferably contains hydrogen and one or more types of gases other than hydrogen. Examples of the gas other than hydrogen include, but are not limited to, carbon dioxide, nitrogen, and carbon monoxide.

The hydrogen concentration of the gas may be 30% or less, preferably 10% or less, and more preferably 4% or less.

### Fermenter, Stirring Power, Pore Size of Sparger

The size of the fermenter is not particularly limited as long as it can accommodate 100 L or more of the culture solution.

When the fermenter is a gas-liquid stirred vessel, the power of a stirrer is not particularly limited as long as it is 0.1 kW/kL or more. When the gas is supplied using a sparger, the pore size of the sparger is not particularly limited.

When the fermenter is a gas-liquid stirred vessel, if the power of the stirrer is less than 0.1 kW/kL, the pore size of the sparger for supplying the gas may be 2 mm or less, preferably 1 mm or less, and more preferably 0.5 mm or less.

When the fermenter is a bubble tower without a stirrer, the pore size of the sparger that supplies the gas may be 2 mm or less, preferably 1 mm or less, and more preferably 0.5 mm or less. If necessary, a draft tube can also be installed.

To efficiently recover the equol, a flow rate of a mixed gas constituting the gas phase to the fermenter is preferably from 0.001 to 2.0 V/V/M gas amount/liquid amount/min.

In the present invention, the temperature of the fermenter is not particularly limited, is preferably a temperature at which the microorganism can exhibit the equol-producing capability, and may be, for example, from 30° C. to 40° C., and preferably from 33° C. to 38° C.

In the present invention, microorganisms can also be cultured at normal pressure, but when microorganisms are to be cultured under pressure, the pressurization condition for cultivation of the microorganisms is not particularly limited as long as the condition allows for growth. Preferable pressurization conditions include, but are not limited to, a range of 0.2 MPa or less.

The fermentation time can be appropriately set according to the production amount of equol, the remaining amount of isoflavones, and the like. The culturing time is, for example, from 8 to 120 hours, preferably from 12 to 72 hours, and particularly preferably from 16 to 60 hours, but is not limited thereto.

A fermentation culture obtained by the culturing method according to an embodiment of the present invention is formed into a solid by heat drying treatment, spray drying treatment, or freeze drying treatment as necessary, and the resultant solid may be used. The heated drying process can be carried out using, for example, a rotary drum dryer, the spray drying process can be carried out using, for example, a spray dryer, and the freeze drying process can be carried out using a freeze dryer. The drying method may use any dryer as long as the dryer can dry the liquid. The dried fermentation culture may be subjected to a grinding process as necessary.

### EXAMPLE

Hereinafter, the present invention will be described on the basis of examples, but the scope of the present invention is not limited to the following examples.

### Example 1

### Preculture 1

A culture medium adjusted to pH 6.9 with the composition shown in Table 1 was dispensed into a 10 mL 18 mm test tube for anaerobic microorganism culture (available from Sanshin Kogyo Co., Ltd.), and sterilized at 121° C. for 15 minutes with a butyl rubber stopper and a plastic cap fitter thereto while the gas phase being purged with nitrogen. Into this culture medium, strain DSM 18785 of Adlercreutzia equolifaciens subsp. celatus was inoculated, and after the gas phase was purged for 2 minutes or more with hydrogen gas which had been passed through a sterile filter, shaking culture was performed at 37° C. and 200 spin for 18 hours to prepare a preculture solution 1.

TABLE 1
Culture medium composition

| Material | Concentration (g/L) |
|---|---|
| Yeast extract | 10.0 |
| Peptone | 5.0 |
| Trypticase peptone | 5.0 |
| Glucose | 5.0 |
| Sodium chloride | 0.08 |
| Dipotassium hydrogen phosphate | 0.04 |
| Potassium dihydrogen phosphate | 0.04 |
| Magnesium sulfate heptahydrate | 0.02 |
| Calcium chloride dihydrate | 0.01 |
| Sodium bicarbonate | 0.4 |
| L-cystine | 0.5 |

### Preculture 2

15 L of the culture medium adjusted to pH 6.9 with the composition shown in Table 1 was placed in a fermenter having a volume of 30 L, heated at 121° C. for 15 minutes to be sterilized with high-pressure steam. The preculture solution 1 was inoculated into this culture medium, and purged with a hydrogen/nitrogen mixed gas, and then cultured at 37° C. for 18 hours to prepare a preculture solution 2.

### Main Culture

To the composition shown in Table 1, 1 g/L of daidzein and 3 g/L of L-arginine were added, and 100 L of the culture medium adjusted to pH 6.9 was placed in a fermenter having a volume of 200 L, and heated at 121° C. for 15 minutes to be sterilized with high-pressure steam. The preculture solution 2 was inoculated into the culture medium, the culture was purged with a mixed gas having each hydrogen/nitrogen ratio, and stirring was performed with each power while the mixed gas was passed through a sparger having each pore size. Culture was performed at 37° C. for 18 hours, and the equol concentration in the present culture solution was analyzed by an HPLC method.

The results are presented in Table 2. From Example 1, it was confirmed that when a gas phase hydrogen concentration was 30% or less, the concentration of equol was remarkably improved by setting a stirring power to 0.1 kW/kL or more. In addition, it was confirmed that in the case of not applying the stirring power, it was confirmed that the equol concentration was improved by setting the sparger pore size to 2 mm or less.

TABLE 2
Results of Example 1

| Gas phase hydrogen | | | | | | | |
|---|---|---|---|---|---|---|---|
| concentration | Other conditions | | | | | | |
| 40% | Stirring power | kW/kL | 0.05 | 0.1 | 0.2 | 0.4 | 1 |
| | Mixed gas flow rate | V/V/min | 0.1 | 0.1 | 0.1 | 0.1 | 0.1 |
| | Fermenter internal pressure | MPa | 0.05 | 0.05 | 0.05 | 0.05 | 0.05 |
| | Sparger pore size | mm | 3 | 3 | 3 | 3 | 3 |
| | Equol concentration | g/L | 0.9 | 0.9 | 0.9 | 0.9 | 0.9 |
| 30% | Stirring power | kW/kL | 0.05 | 0.1 | 0.2 | 0.4 | 1 |
| | Mixed gas flow rate | V/V/min | 0.1 | 0.1 | 0.1 | 0.1 | 0.1 |
| | Fermenter internal pressure | MPa | 0.05 | 0.05 | 0.05 | 0.05 | 0.05 |
| | Sparger pore size | mm | 3 | 3 | 3 | 3 | 3 |
| | Equol concentration | g/L | 0.2 | 0.5 | 0.9 | 0.9 | 0.9 |
| 10% | Stirring power | kW/kL | 0.05 | 0.1 | 0.2 | 0.4 | 1 |
| | Mixed gas flow rate | V/V/min | 0.1 | 0.1 | 0.1 | 0.1 | 0.1 |
| | Fermenter internal pressure | MPa | 0.05 | 0.05 | 0.05 | 0.05 | 0.05 |
| | Sparger pore size | mm | 3 | 3 | 3 | 3 | 3 |
| | Equol concentration | g/L | 0 | 0.4 | 0.8 | 0.9 | 0.9 |
| 4% | Stirring power | kW/kL | 0.05 | 0.1 | 0.2 | 0.4 | 1 |
| | Mixed gas flow rate | V/V/min | 0.1 | 0.1 | 0.1 | 0.1 | 0.1 |
| | Fermenter internal pressure | MPa | 0.05 | 0.05 | 0.05 | 0.05 | 0.05 |
| | Sparger pore size | mm | 3 | 3 | 3 | 3 | 3 |
| | Equol concentration | g/L | 0 | 0.3 | 0.6 | 0.9 | 0.9 |
| 4% | Stirring power | kW/kL | 0.05 | 0.2 | 0 | 0 | 0 |
| | Mixed gas flow rate | V/V/min | 0.01 | 0.01 | 0.5 | 0.5 | 0.3 |
| | Fermenter internal pressure | MPa | 0.1 | 0.1 | 0.02 | 0.02 | 0.02 |
| | Sparger pore size | mm | 3 | 3 | 3 | 1 | 1 |
| | Equol concentration | g/L | 0 | 0.6 | 0 | 0.6 | 0.6 |
| 4% | Stirring power | kW/kL | 0 | 0 | 0 | 0 | 0 |
| | Mixed gas flow rate | V/V/min | 0.1 | 0.1 | 0.1 | 0.1 | 0.05 |
| | Fermenter internal pressure | MPa | 0.05 | 0.05 | 0.05 | 0.05 | 0.1 |
| | Sparger pore size | mm | 3 | 2 | 1 | 0.5 | 0.5 |
| | Equol concentration | g/L | 0 | 0.3 | 0.7 | 0.9 | 0.9 |

### Example 2

### Preculture 1

A culture medium adjusted to pH 6.9 with the composition shown in Table 1 was dispensed into a 10 mL 18 mm test tube for anaerobic microorganism culture (available from Sanshin Kogyo Co., Ltd.), and sterilized at 121° C. for 15 minutes with a butyl rubber stopper and a plastic cap fitter thereto while the gas phase being purged with nitrogen. Into this culture medium, strain DSM 18785 of Adlercreutzia equolifaciens subsp. celatus was inoculated, and after the gas phase was purged for 2 minutes or more with hydrogen gas which had been passed through a sterile filter, shaking culture was performed at 37° C. and 200 spin for 18 hours to prepare a preculture solution 1.

### Preculture 2

15 L of the culture medium adjusted to pH 6.9 with the composition shown in Table 1 was placed in a fermenter having a volume of 30 L, heated at 121° C. for 15 minutes to be sterilized with high-pressure steam. The preculture solution 1 was inoculated into this culture medium, and purged with a hydrogen/nitrogen mixed gas, and then cultured at 37° C. for 18 hours to prepare a preculture solution 2.

### Preculture 3

100 L of the culture medium adjusted to pH 6.9 with the composition shown in Table 1 was placed in a fermenter having a volume of 200 L, heated at 121° C. for 15 minutes to be sterilized with high-pressure steam. The preculture solution 2 was inoculated into this culture medium, and purged with a hydrogen/nitrogen mixed gas, and then cultured at 37° C. for 18 hours to prepare a preculture solution 3.

### Main Culture

To the composition shown in Table 1, 1 g/L of daidzein and 3 g/L of L-arginine were added, and 2000 L of the culture medium adjusted to pH 6.9 was placed in a fermenter having a volume of 4000 L, and heated at 121° C. for 15 minutes to be sterilized with high-pressure steam. The preculture solution 3 was inoculated into the culture medium, purged with a nitrogen mixed gas having a hydrogen concentration of 4%, and stirred with each power while the mixed gas was passed through a sparger having a pore size of 3 mm. Culture was performed at 37° C. for 18 hours, and the equol concentration in the present culture solution was analyzed by an HPLC method.

The results are presented in Table 3. From Example 2, it was confirmed that even when the size of the fermenter was increased, the concentration of equol was remarkably improved by setting the stirring power to 0.1 kW/kL or more.

TABLE 3
Results of Example 2

| Gas phase hydrogen | | | | | | | |
|---|---|---|---|---|---|---|---|
| concentration | Other conditions | | | | | | |
| 4% | Stirring power | kW/kL | 0.05 | 0.1 | 0.2 | 0.4 | 1 |
| | Mixed gas flow rate | V/V/min | 0.1 | 0.1 | 0.1 | 0.1 | 0.1 |
| | Fermenter internal pressure | MPa | 0.05 | 0.05 | 0.05 | 0.05 | 0.05 |
| | Sparger pore size | mm | 3 | 3 | 3 | 3 | 3 |
| | Equol concentration | g/L | 0 | 0.3 | 0.6 | 0.9 | 0.9 |

### Example 3

### Preculture 1

A culture medium adjusted to pH 6.9 with the composition shown in Table 1 was dispensed into a 10 mL 18 mm test tube for anaerobic microorganism culture (available from Sanshin Kogyo Co., Ltd.), and sterilized at 121° C. for 15 minutes with a butyl rubber stopper and a plastic cap fitter thereto while the gas phase being purged with nitrogen. An Eggerthella sp. DC 3215 strain was inoculated into this culture medium, the gas phase was purged for 2 minutes or more with hydrogen gas which had been passed through a sterile filter, and then shaking culture was performed at 37° C. and 200 spin for 36 hours to prepare a preculture solution 1.

### Preculture 2

15 L of the culture medium adjusted to pH 6.9 with the composition shown in Table 1 was placed in a fermenter having a volume of 30 L, heated at 121° C. for 15 minutes to be sterilized with high-pressure steam. The preculture solution 1 was inoculated into this culture medium, and purged with a hydrogen/nitrogen mixed gas, and then cultured at 37° C. for 36 hours to prepare a preculture solution 2.

### Main Culture

To the composition shown in Table 1, 0.5 g/L of daidzein and 3 g/L of L-arginine were added, and 100 L of the culture medium adjusted to pH 6.9 was placed in a fermenter having a volume of 200 L, and heated at 121° C. for 15 minutes to be sterilized with high-pressure steam. The preculture solution 2 was inoculated into the culture medium, purged with a nitrogen mixed gas having a hydrogen concentration of 4%, and stirred with each power while the mixed gas was passed through a sparger having a pore size of 3 mm. Culture was performed at 37° C. for 72 hours, and the equol concentration in the present culture solution was analyzed by an HPLC method.

The results are presented in Table 4. From Example 3, it was confirmed that equol was produced by setting a stirring power to 0.1 kW/kL or more.

TABLE 4
Results of Example 3

| Gas phase | | | | | |
|---|---|---|---|---|---|
| hydrogen | | | | | |
| concentration | Other conditions | | | | |
| 4% | Stirring power | kW/kL | 0.05 | 0.1 | 1 |
| | Mixed gas flow rate | V/V/min | 0.02 | 0.02 | 0.02 |
| | Fermenter internal | MPa | 0.05 | 0.05 | 0.05 |
| | pressure | | | | |
| | Sparger pore size | mm | 3 | 3 | 3 |
| | Equol concentration | g/L | 0 | 0.1 | 0.1 |

### Example 4

### Preculture 1

A culture medium adjusted to pH 6.5 with the composition shown in Table 1 was dispensed into a 10 mL 18 mm test tube for anaerobic microorganism culture (available from Sanshin Kogyo Co., Ltd.), and sterilized at 121° C. for 15 minutes with a butyl rubber stopper and a plastic cap fitter thereto while the gas phase being purged with nitrogen. A Lactococcus sp. DCL strain was inoculated into this culture medium, the gas phase was purged for 2 minutes or more with hydrogen gas which had been passed through a sterile filter, and then shaking culture was performed at 37° C. and 200 spin for 24 hours to prepare a preculture solution 1.

### Preculture 2

15 L of the culture medium adjusted to pH 6.5 with the composition shown in Table 1 was placed in a fermenter having a volume of 30 L, heated at 121° C. for 15 minutes to be sterilized with high-pressure steam. The preculture solution 1 was inoculated into this culture medium, and purged with a hydrogen/nitrogen mixed gas, and then cultured at 37° C. for 24 hours to prepare a preculture solution 2.

### Main Culture

50 g/L of powdery soybean hypocotyls, 3 g/L of L-arginine, and 2 g/L of soybean oil containing vitamin E were added, and 100 L of the culture medium adjusted to pH 6.5 was placed in a fermenter having a volume of 200 L, and heated at 121° C. for 15 minutes to be sterilized with high-pressure steam. The preculture solution 2 was inoculated into the culture medium, purged with a nitrogen mixed gas having a hydrogen concentration of 4%, and stirred with each power while the mixed gas was passed through a sparger having a pore size of 3 mm. Culture was performed at 37° C. for 96 hours, and the equol concentration in the present culture solution was analyzed by an HPLC method.

The results are presented in Table 5. From Example 4, it was confirmed that equol was produced by setting a stirring power to 0.1 kW/kL or more.

TABLE 5
Results of Example 4

| Gas phase | | | | | |
|---|---|---|---|---|---|
| hydrogen | | | | | |
| concentration | Other conditions | | | | |
| 4% | Stirring power | kW/kL | 0.05 | 0.1 | 1 |
| | Mixed gas flow rate | V/V/min | 0.2 | 0.2 | 0.2 |
| | Fermenter internal | MPa | 0.07 | 0.07 | 0.07 |
| | pressure | | | | |
| | Sparger pore size | mm | 3 | 3 | 3 |
| | Equol concentration | g/L | 0 | 0.2 | 0.3 |

1. An equol manufacturing method for producing equol comprising fermenting at least one type of equol raw material selected from the group consisting of daidzein glycoside, daidzein, and dihydrodaidzein, under a gas phase including one or more types of gases containing hydrogen, by a microorganism that assimilates the equol raw material to produce equol,
wherein in the fermentation, a fermenter having a culture solution in an amount of 100 L or more is used, and
i) stirring is performed at a stirring power of 0.1 kW/kL or more in the fermenter; and/or
ii) the one or more types of gases are introduced by using a sparger having a pore size of 2 mm or less.

2. The method according to claim 1, wherein the stirring power is 0.2 kW/kL or more.

3. The method according to claim 1, wherein the stirring power is 0.4 kW/kL or more.

4. The method according to claim 1, wherein the sparger has a pore size of 1 mm or less.

5. The method according to claim 1, wherein the sparger has a pore size of 0.5 mm or less.

6. The method according to claim 1, wherein the one or more types of gases contain a hydrogen at a concentration of 30% or less.

7. The method according to claim 1, wherein the one or more types of gases contain a hydrogen at a concentration of 10% or less.

8. The method according to claim 1, wherein the one or more types of gases contain a hydrogen at a concentration of 4% or less.

9. The method according to claim 4, wherein the one or more types of gases contain a hydrogen at a concentration of 30% or less.

10. The method according to claim 4, wherein the one or more types of gases contain a hydrogen at a concentration of 10% or less.

11. The method according to claim 4, wherein the one or more types of gases contain a hydrogen at a concentration of 4% or less.

12. The method according to claim 5, wherein the one or more types of gases contain a hydrogen at a concentration of 30% or less.

13. The method according to claim 5, wherein the one or more types of gases contain a hydrogen at a concentration of 10% or less.

14. The method according to claim 5, wherein the one of more types of gases contain a hydrogen at a concentration of 4% or less.
